(19) United States
(12) Patent Application Publication
(10) Pub. No.: US 2012/0283575 A1
(43) Pub. Date: Nov. 8, 2012

(54) REMOTE NON-INVASIVE PARAMETER SENSING SYSTEM AND METHOD

(76) Inventors: Govind Rao, Ellicott City, MD (US); Yordan Kostov, Columbia, MD (US); Leah Tolosa, Columba, MD (US); Hung Lam, Baltimore, MD (US)

(21) Appl. No.: 13/501,081

(22) PCT Filed: Oct. 12, 2010
(86) PCT No.: PCT/US10/52286

§ 371 Date: Jul. 23, 2012

Related U.S. Application Data
(60) Provisional application No. 61/250,049, filed on Oct. 9, 2009.

(51) Int. Cl. A61B 5/01; A61B 6/00

(52) U.S. Cl. 600/476

(57) ABSTRACT

A remote parameter sensing system is provide that includes a gel sensor, a light source, a detector and a controller 140. The gel sensor is in contact with a surface where the parameter is to be measured, and is preferably a gel that is embedded with a chemical that emits light 160 (via, for example, fluorescence) when it is excited by excitation light from the light source at an appropriate excitation frequency. The chemical properties of the gel sensor are such that at least one characteristic of the emission light (such as, for example, emission intensity) varies as a function of variations in the parameter being measured. The system is particularly suited for use as remote body temperature sensing system in incubators and radiant warmers for infant and neonatal care.

This application claims priority to U.S. Provisional Application Ser. No. 61/250,049, filed Oct. 9, 2009, whose entire disclosure is incorporated herein by reference.

### BACKGROUND OF THE INVENTION

1. Field of the Invention

The present invention relates to remote sensing of predetermined parameters such as, for example, remote sensing of human body temperature.

2. Background of the Related Art

The Background of the Related Art and the Detailed Description of Preferred Embodiments below cite numerous technical references, which are listed in the Appendix below. The numbers shown in parenthesis at the end of some of the sentences refer to specific references listed in the Appendix. For example, a “(1)” shown at the end of a sentence refers to reference “1” in the Appendix below. All of the references listed in the Appendix below are incorporated by reference herein in their entirety.

Internal body temperature is a physiologic variable that is precisely controlled by the body. Chemical processes and enzymes required to catalyze the associated chemical reactions, thereby regulating cellular function, optimally operate within this narrow thermal bandwidth. Thermoregulation encompasses all physiological processes and responses that balance heat production and heat loss to maintain body temperature within this normal range. Compared to the adult or older pediatric model, thermoregulation is even more critical to neonatal care (1).

Newly born infants have a limited ability to achieve chemical and physical thermal homeostasis during transition from intra- to extrauterine life due to physiologic differences in bodily function and small body size, which accounts for this vulnerability (2). Infants that cannot maintain their body temperature within a fairly narrow range after birth, for whatever reason, may die in the absence of a warming device. Hence, maintaining a neutral thermal environment (NTE) is a cornerstone of immediate caregiving for this population. NTE is the ambient temperature at which oxygen consumption and energy expenditure is at a minimum to sustain vital bodily functions described above. A baby's ideal ambient temperature varies depending on a baby's gestational and postnatal age, as well as a variety of clinical factors (3, 4).

Compared to adults, newborns are particularly vulnerable to heat loss. These losses may be incurred across four partitions: radiation, convection, conduction, or evaporation. The importance of each partition changes across the infant's life depending upon his or her gestational and postnatal age. Additionally, other clinical factors such as weight and acuity of illness also impact the NTE. If losses across these partitions are not pre-empted, newborns may be subjected to the effects of cold stress or hypothermia. In fact, among certain gestations, mortality increases by 10% for each degree Celsius that a baby's body temperature is below 36° C. (5). Conversely, if heat is not needed or is supplied in an uncontrolled manner, these infants may also be subjected to the dangers of heat stress or hyperthermia. Both types of thermal stresses, hypothermia and hyperthermia, can be a significant source of morbidity and mortality in this vulnerable population (5, 6).

Heat can be supplied by convection, radiation, or conduction as a means to counterbalance heat losses experienced by newborns. The modern history of neonatal temperature control began in the late 19th century with the observation by Pierre Budin at the Paris Maternity Hospital that mortality rates decreased from 66% to 38% in infants under 2000 g at birth, following introduction of temperature control measures. These measures involved use of incubators heated through a variety of methods to keep the neonate warm.

In 1957, Silverman reported that use of relative humidity in these enclosed microenvironments further enhanced survival of preterm infants in the first days of life. During this study, it was noted that the mean body temperature of infants maintained in humidified environments was significantly higher than the mean body temperature of infants in incubators with lower relative humidity (30% to 60%). This observation led to the formulation of the normothermic hypothesis, which states that the survival of preterm infants is favorably influenced by environments that maintain normal body temperature.

Radiation is the transfer of heat between two solid objects not in direct contact. Heat energy is transferred by electromagnetic infrared (IR) waves in the far IR (>2.0 μm) range. It has been shown this form of radiant energy can penetrate 0.2 to 0.4 mm below the human skin surface. Therefore, a baby's epidermis absorbs nearly all the IR energy and converts it to heat that may then be transferred to deeper tissues by conductive means through solid organs in direct contact with each other and by convective methods through blood circulation (6). Radiant warmers were invented as an alternative to incubators as the acuity of illness among newborns increased. These devices have been in the commercial healthcare mainstream for approximately 50 years and are particularly useful in the labor and delivery setting during transition to extrauterine life and during resuscitation. Additionally, the device has gained broad acceptance in the neonatal intensive care setting at the point of admission when many procedural interventions must be performed to facilitate procedural access and recovery from illness (2).

There are three main components of a radiant warmer: the bed platform (architectural component), the IR energy output device (heat engine component), and the control algorithm (software component) for the IR energy output. Similarly, there are three main components of an incubator: the bed platform (architectural component), the convective energy output device (heat sink and fan component), and the control algorithm (software component) for the convective energy output.

Several modes of temperature control in infant incubators and radiant warmers are used to regulate the heater power output. Most modern microenvironments allow the caregiver to choose between skin temperature servocontrol (incubator and radiant warmer), air temperature servocontrol (incubator only), and manual (nonservo) control (radiant warmer only). With skin servocontrol, heater power output automatically adjusts to changes in the temperature of the infant's skin. Air temperature servocontrol acts the same as skin temperature servocontrol, but the controlling variable is the temperature of the air. Manual control requires human intervention to maintain the desired temperature. A setting is changed in response to intermittent measurement of skin or air temperature. Manual control is seldom used in modern neonatology (6, 7).

Throughout the world, glass or electronic thermometers remain the most common method of temperature measurement in healthy term infants, although newer electronic thermometers are becoming increasingly popular. These measurement tools are generally accurate and inexpensive and are used for routine clinical measurements in which single point determinations are sufficient. However, the need to measure skin or air temperatures continuously to servocontrol heater power outputs for, either an incubator or a radiant warmer, for environmental temperature control has resulted in the clinical introduction of various temperature transducers. The most widely used device to measure and control the thermal environment in newborns is the thermal resistor (thermistor) (7).

A thermistor is a semiconductor that has a large coefficient of resistance. Most thermistors are made from combinations of metal oxides (e.g., manganese, nickel, or copper). They are usually of the negative thermal coefficient type, which exhibits a drop in resistance when the temperature rises. When a thermistor is operated at a power level that is low enough to produce insignificant self-heating, it is referred to as a zero-power resistor. For temperature measurement, the resistance is measured over a resistance bridge where two resistances are known (7).

At present, no data exist that demonstrate which servocontrol mode is the best in radiant warmers and incubators. Skin servocontrol keeps the baby's skin temperature constant at all times. Changes in humidity, air currents, or wall temperature will have a smaller effect on the baby's skin temperature when compared with constant heater output (manual control). However, dislodgment of the probe or accidental placement of the thermistor between the body and the mattress may result in over- or underheating, respectively. In addition, potentially large fluctuations in air temperature may have negative side effects (e.g., apnea). Use of skin temperature servocontrol loses a major sign of disease (i.e., fever). Air temperature servocontrol, on the other hand, produces a more stable environment, but the patient is omitted from the thermal feedback loop (6, 7).

In order for skin servocontrol to occur in modern devices, it is necessary to attach the thermistor to the infant's skin. The current accepted practice is to use a single point measurement with a thermistor affixed to the torso with an adhesive. However, thermogenic images have shown that there is considerable variation in skin temperatures across the body surface. Ideally, an ensemble average from multiple spots would be desirable, but affixing several wired thermistors is undesirable and impractical.

No matter what site is selected, an adhesive-based method us currently used to make the connection between the baby and the bed. Adhesive use on temperature thermistor probe covers is not an innocuous intervention. Investigators have found increased microbial growth beneath probe covers, some of which have proved to be pathogenic. Others have found skin impairment ranging from chemical sensitivities to prolonged mechanical force on the skin from some adhesives. Further, adhesives can irritate the skin by occlusion or by altering the skin morphology via epidermal stripping. “Skin tears” associated with adhesive removal as a skin temperature probe cover is removed results from shear or frictional forces that separate the dermis from the epidermis. This compromises skin barrier function and a marked increases transpidermal water loss. In many cases, the skin can no longer protect against microorganism invasion.

Thermogenic imaging cameras are simply too expensive to be a viable solution. Furthermore, the radiant warmers employed would interfere with their operation. Accordingly, there is a great need for more accurate temperature sensing that is preferably remote and non-invasive.

### SUMMARY OF THE INVENTION

An object of the invention is to solve at least the above problems and/or disadvantages and to provide at least the advantages described hereinafter.

Therefore, an object of the present invention is to provide a system and method for the remote sensing of a parameter, such as temperature, in a non-invasive and/or non-contact manner.

Another object of the present invention is to provide a system and method for remotely measuring body temperature in a non-invasive and/or non-contact manner.

Another object of the present invention is to provide a system and method for remotely measuring the body temperature of an infant in a non-invasive and/or non-contact manner.

To achieve at least the above objects, in whole or in part, there is provided a remote sensor for measuring a parameter of a system, comprising a light source for generating excitation light, a gel sensor in physical communication with the system and positioned to receive the excitation light, wherein the gel sensor emits emission light in response to the excitation light and wherein a chemical property of the gel sensor is such that at least one characteristic of the emission light varies as a function of variations in the parameter being measured, a detector for detecting the emission light from the gel sensor and for outputting a detector signal based on the detected emission light, and a controller for receiving and analyzing the detector signal and for deriving a parameter value based on the detector signal.

To achieve at least the above objects, in whole or in part, there is also provided a system for remotely monitoring body temperature, comprising a light source for generating excitation light, a gel sensor in physical contact with the body and positioned to receive the excitation light, wherein the gel sensor emits emission light in response to the excitation light and wherein a chemical property of the gel sensor is such that at least one characteristic of the emission light varies as a function of temperature, a detector for detecting the emission light from the gel sensor and for outputting a detector signal based on the detected emission light, and a controller for receiving and analyzing the detector signal and for deriving a body temperature based on the analysis.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIG. 1 is a schematic diagram of a remote sensor for measuring a parameter of a system, in accordance with the present invention;

FIG. 2 is a system for remotely monitoring body temperature, in accordance with the present invention;

FIG. 3 is a plot of the emission spectrum of rubpy;

FIG. 4 is a plot of the emission intensity of rubpy as a function of temperature;

FIG. 5 is a plot of the luminance decay time of rubpy as a function of temperature;

FIG. 6 is a plot illustrating the repeatability of the emission response in rubpy that has been entrapped in polyacrylonitrile;

FIG. 7 is a plot of the absorption spectrum of eutdap as a function of temperature;

FIG. 8A is a plot of the emission spectrum of eutdap as a function of temperature;

FIG. 8B is a plot of the luminance decay time of eutdap as a function of temperature;

FIG. 9 is a schematic diagram of an LED light source used in one embodiment of the present invention;

FIG. 10 is shows an example of a CCD camera that can be used as a detector in one embodiment of the present invention; and

FIG. 11 is a schematic diagram of an incubator/radiant warmer that incorporates a remote body temperature sensing system, in accordance with one embodiment of the present invention.

### DETAILED DESCRIPTION OF PREFERRED EMBODIMENTS

By way of example, the present invention will be described in connection with a remote temperature sensing system and method that is particularly suited for the remote sensing of skin temperature in infants. However, it should be appreciated that the present invention can be used as a remote sensor for other types of parameters such as, for example, CO2, pH, ammonia, oxygen, sodium, calcium and potassium.

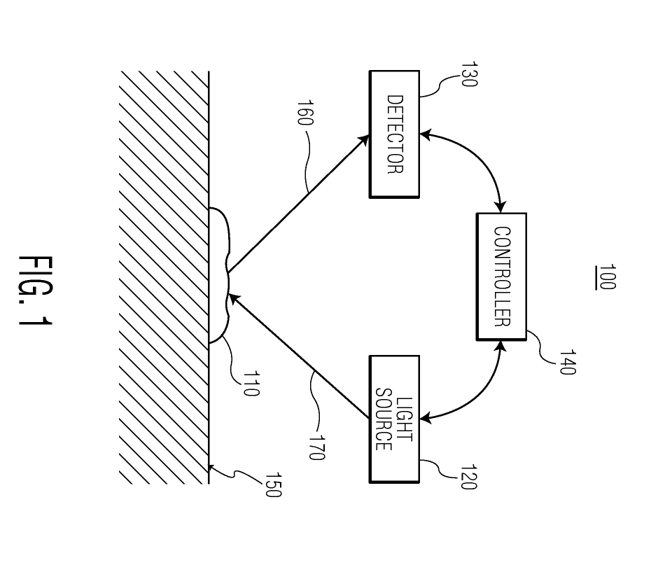

FIG. 1 is a schematic diagram of a remote parameter sensing system 100, in accordance with one embodiment of the present invention. The remote parameter sensing system 100 includes a gel sensor 110, a light source 120, a detector 130 and a controller 140. The gel sensor 110 is in contact with a surface 150 where the parameter is to be measured, and is preferably a gel that is embedded with a chemical that emits light 160 (via, for example, fluorescence) when it is excited by excitation light 170 from the light source 120 at an appropriate excitation frequency. The chemical properties of the gel sensor 110 are such that at least one characteristic of the emission light 160 (such as, for example, emission intensity) varies as a function of variations in the parameter being measured.

The controller 140 controls the measurement process, including control of the light source 120, receiving detector signals from the detector 130 and processing and analyzing the detector signals to analyze the parameter being measured.

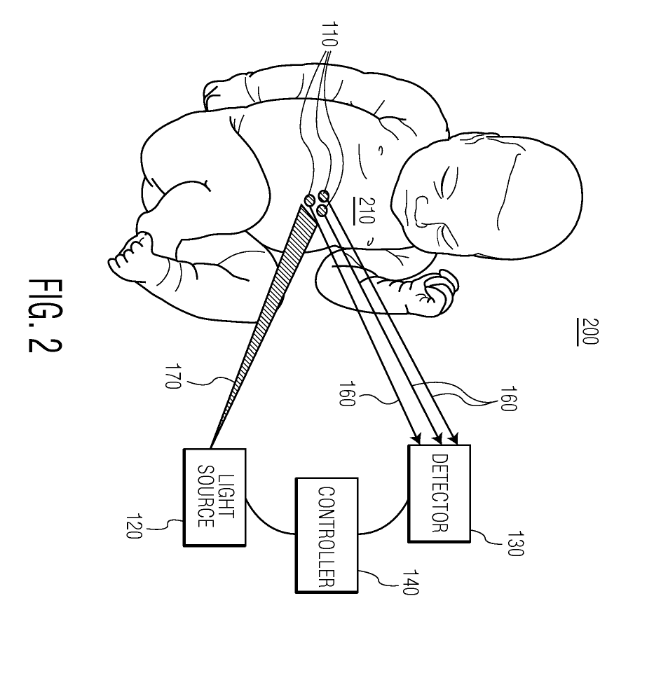

FIG. 2 is a system for remotely monitoring body temperature 200, in accordance with another embodiment of the present invention. The system includes a light source 120, detector 130, controller 140, and at least one gel sensor 110 placed on the skin 210 of the subject whose temperature is being measured.

The gel sensors 110 are preferably temperature sensitive nontoxic luminophores in a hydrogel matrix. The luminescence intensity and/or the luminescence decay time of the temperature sensitive nontoxic luminophores preferably vary as a function of temperature.

The gel sensors 110 are preferably applied onto multiple sites on the subject's skin 210. This gel sensors are illuminated from a distance with excitation light 170 from light source 120, and the excitation light 160 is detected by detector 130. The detector signals are then analyzed by controller 140. In one preferred embodiment, the detector 130 is a movable CCD camera and the controller 140 is programmed with shape recognition software for controlling the position of the CCD camera to follow the gel sensors 110 as the subject moves.

The temperature sensing system 200 offers many advantages of over existing systems. First, there is no need for problematic adhesives to keep the gel sensors 110 in place. It is very easy to daub the gel sensors 110 on the skin 210, and the gel sensors 110 can be removed easily by gentle wiping with a wet tissue. Thus, the skin 210 will not be exposed to any stress associated with applying and removing an adhered probe.

Another advantage is the flexibility of the gel sensors 110. For example, gel sensors 110 can be smeared anywhere and at multiple points on the subject. In contrast, taking temperature readings at multiple points with wired thermistors can be very cumbersome, and creates the added danger of an infant getting caught in a tangle of wires. Further, as the gel sensors 110 are always in close contact with the skin 210, optimal heat transfer from the skin 210 to the gel sensors 110 are ensured at all times, thereby increasing the accuracy of the temperature measurement and its correlation to core body temperature.

While temperature sensing luminophores have been used in the art, they require UV light excitation, which is not acceptable for infants, or they emit light in the IR wavelength range where radiant warmers will interfere. The gel sensors 110 in system 200 are preferably designed to be excited with and emit light in the visible wavelength range.

### Gel Sensors 110

As discussed above, the gel sensors 110 are preferably temperature sensitive nontoxic luminophores in a hydrogel matrix. The preferred properties of the luminophores and hydrogel matrix will now be discussed.

### Temperature Sensitive Luminophores

Luminescence based temperature sensing has been widely investigated. A variety of luminophores have been found to show high sensitivity to temperature and they have been utilized as luminescent temperature probes (9). However, these luminophores are not suitable for neonatal healthcare. As an example, alexandrite crystals were found to be sensitive between 15-45° C., which is the right range of temperatures for human temperature monitoring, and the phosphorescence lifetime decreases from 300 to 220 μs within this temperature range. However, alexandrite crystals cannot be ground to a fine powder for application on infant skin. The grinding process creates defects in the crystal structure, rendering alexandrite non-luminescent.

Zinc sulfide shows strong temperature sensitivity between 25 to 50° C., and Lanthanide phosphors, such as La₂O₂S:Eu, are also responsive to temperature changes over a wide range. The lifetime of La₂O₂S:Eu decreases over an order of magnitude as the temperature increases from 0 to 100° C. (10). However, all of these luminophores are only excitable by UV light, which is potentially harmful to neonate skin.

Among the luminophores that are excitable by non-UV light are ruthenium(II)tris(1,10-phenanthroline) (ruphen), ruthenium(II) tris(bipyridine) (“rubpy”) and Tris- (dibenzoylmethane) mono(5-amino-1,10-phenanthroline)-europium(III) (“eutdap”). These luminophore dyes are not only highly luminescent, but their luminescence intensity and decay time are also very sensitive to temperature. Moreover, they are found to be nontoxic and incapable of penetrating human skin (11).

Due to all the advantages of luminescence sensors described above, several luminescence-based temperature probes have been developed. In general, there are three approaches for measuring temperature: (1) steady state luminescence intensity measurements; (2) ratiometric intensity measurements; and (3) decay time measurements. The simplest is approach (1), which measures the steady state luminescence intensity. However, this approach is not suitable for long term measurements, as the intensity can drift due to photobleaching of the luminophore. Methods (2) and (3) offer solutions to this problem.

Approach (2) takes the intensity ratio of two emission bands of the luminophore system to represent the temperature. Assuming the photobleaching effect has the same impact on the emission bands, the ratio is unaffected, thereby making the system less prone to drift.

Approach (3) utilizes the decay time of the luminophore as the temperature sensitive parameter. The decay time is the time span required for the luminophore dye at the excited state to return to the electronic ground state. This transition is temperature dependent. Since it is an intrinsic property of the dye molecule, the temperature measurement based on the decay time technique is entirely independent of the luminophore concentration.

The luminophore dyes mentioned above have been widely employed as temperature probes in various research fields. Luminescence based temperature probes have a variety of advantages over thermoelectric probes. These include their virtually unlimited spatial resolution, the immunity to high electromagnetic fields and the capability for long distance measurements. For example, luminophores can be employed in thermal convection studies in both huge bioreactors and micro-sized lab-on-a-chip (12). Further, since light transmission requires no conducting medium, the probe and the photodetector need not to be in direct contact. Accordingly, remote detection can be realized.

The luminescence process is initiated by the absorption of light by the ground state luminophore, and in the process promoting the molecule to an electronic excited state. The list of ruthenium luminophores mentioned above undergo intersystem crossing from the lowest singlet excited state to the triplet state (technically a singlet-triplet hybrid), which is normally at a lower energy. Return of the triplet to the singlet ground state requires intersystem crossing, i.e. a “forbidden” process. This explains both the long decay lifetimes, as well as the red-shifted emission.

It should be mentioned that the triplet state, because of its same spin electronic configuration, is particularly susceptible to quenching by oxygen. In the presence of oxygen, the excited dye can transfer the absorbed energy to the oxygen which is then transformed to the very reactive singlet oxygen. In this excited form, oxygen is chemically very reactive and can oxidize (bleach) the luminophore (13). Thus, discussed above, the sensing dye has to be protected from oxygen in order to preserve its integrity and functionality. In one preferred embodiment, the dye is preferably protected from oxygen by embedding it in a transparent polymer with extremely low oxygen permeability. A suitable polymer is polyacrylonitrile (PAN), which has an oxygen permeability of 1.5*10⁻⁴ cm³ cm cm⁻² s⁻¹ Pa⁻¹ (14).

Of the luminophores listed above, two preferable choices for use in the present invention are rubpy and eutdap. Rubpy is a highly luminescent complex and is photochemically very stable. The excitation peak of rubpy is found at 455 nm and can be excited with standard commercially available blue LEDs.

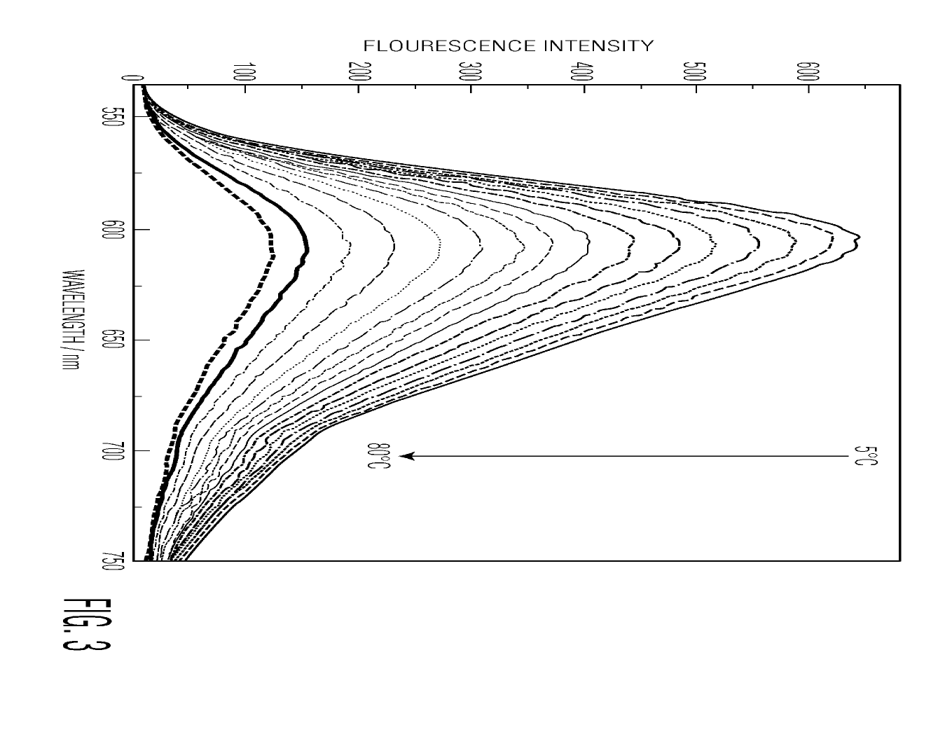

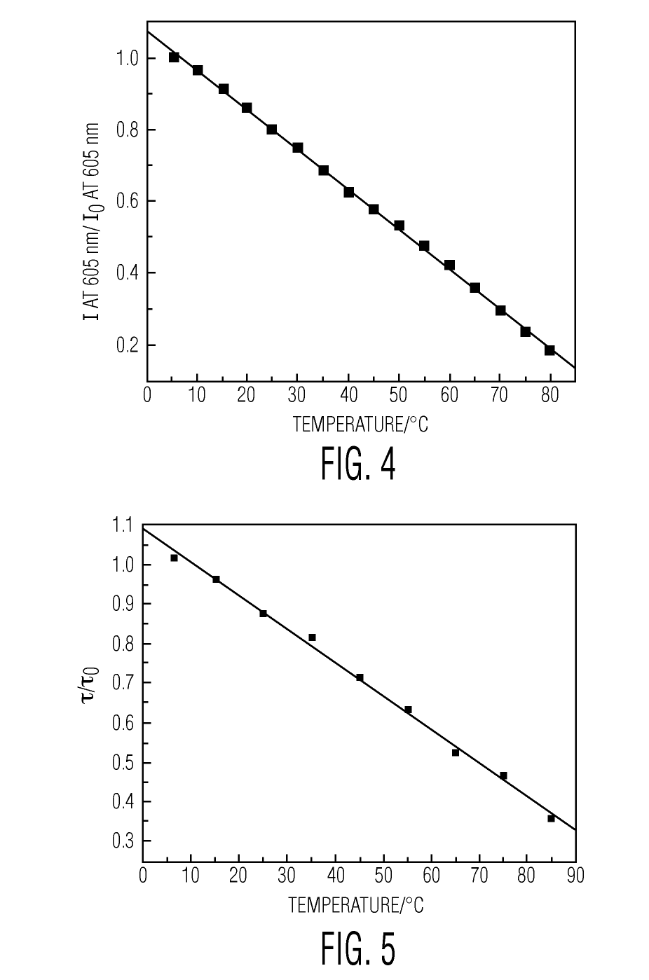

During testing, rubpy was entrapped in a PAN film with a thickness of less than 30 μm. The luminescence peak is centered at around 605 nm, as shown in FIG. 3. Due to the large Stokes shift, the excitation and emission light can be easily separated by an optical long pass or band pass filter. Spectroscopic measurements reveal the high temperature sensitivity of rubpy. As shown in FIG. 4, the luminescence intensity decreases about 90 percent when the temperature is increased from 0 to 80° C. The emission intensity change is linear within this range.

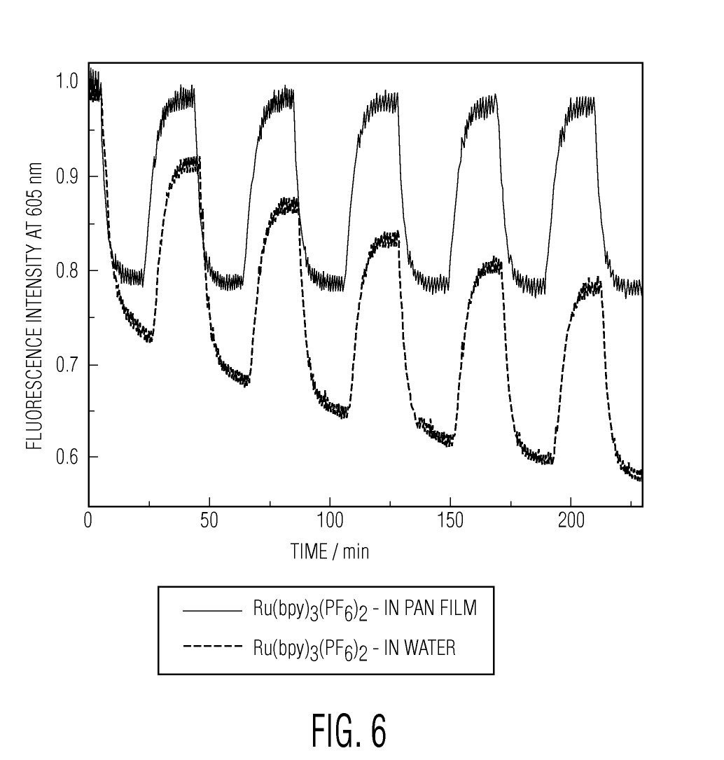

FIG. 5 shows that the luminescence decay time decreases linearly with the temperature 1.8 to 0.7 μs. As can be seen in FIG. 6, the entrapment of rubpy in PAN greatly improves the stability of luminescence emission. Continuous measurement for 4 hours diminishes the luminescence intensity by 25 percent of the initial intensity when dissolved in water. In contrast, the luminescence intensity of rubpy entrapped in PAN remains practically unchanged. This suggests that the chemical integrity of the dye is significantly protected by the polymer from the destructive oxygen in the environment.

The other preferred luminophore is eutdap. Europium ions are intrinsically luminescent like many other lanthanide ions. However, the luminescence intensity is greatly improved by forming complexes with ligands, which serve as light antennas.

The central europium ion is excited through charge transfer processes from the outer ligands. Luminescence occurs when the excited europium ion returns to its ground state by an f-f transition. Since this transition is “forbidden”, the return to the ground state takes considerably longer than “allowed” electronic transitions. This results in a much longer decay time. In fact, eutdap exhibits a decay time of more than 300 μs when incorporated in PAN. From the point of view of instrument design, the long decay time is a very attractive feature, as it can be easily and more precisely determined with less sophisticated instrumentation.

Europium and all other lanthanide chelates differ from other metal complexes in that their valence orbitals (4f orbital) are not the outermost, but are shielded by the 5s, 5p, 5d and 6s orbitals. Thus, the surrounding environment has a low impact on the center ion of these complexes.

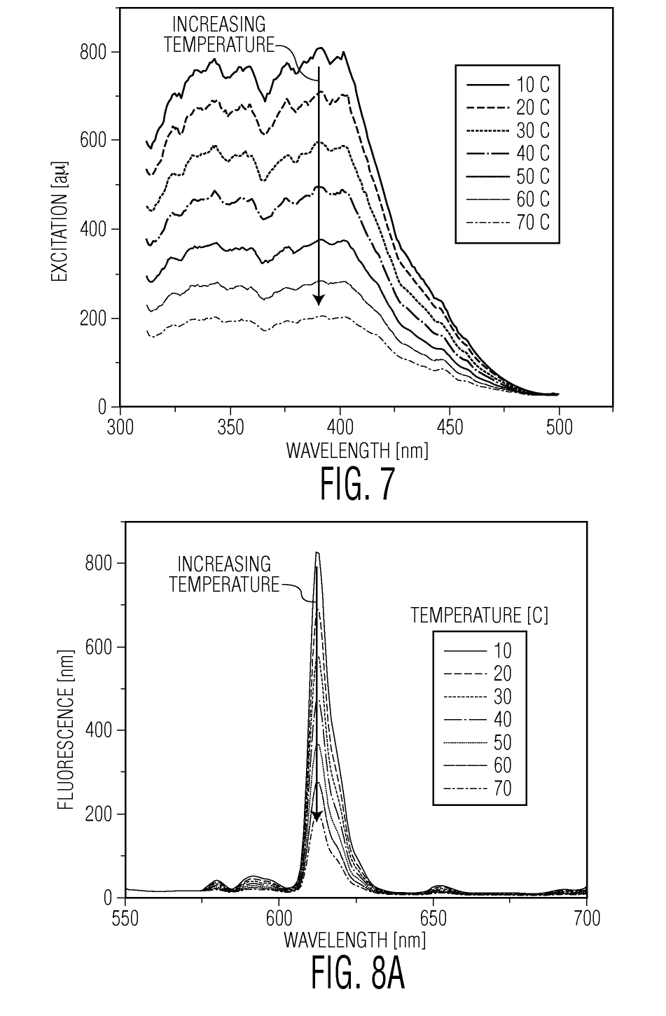

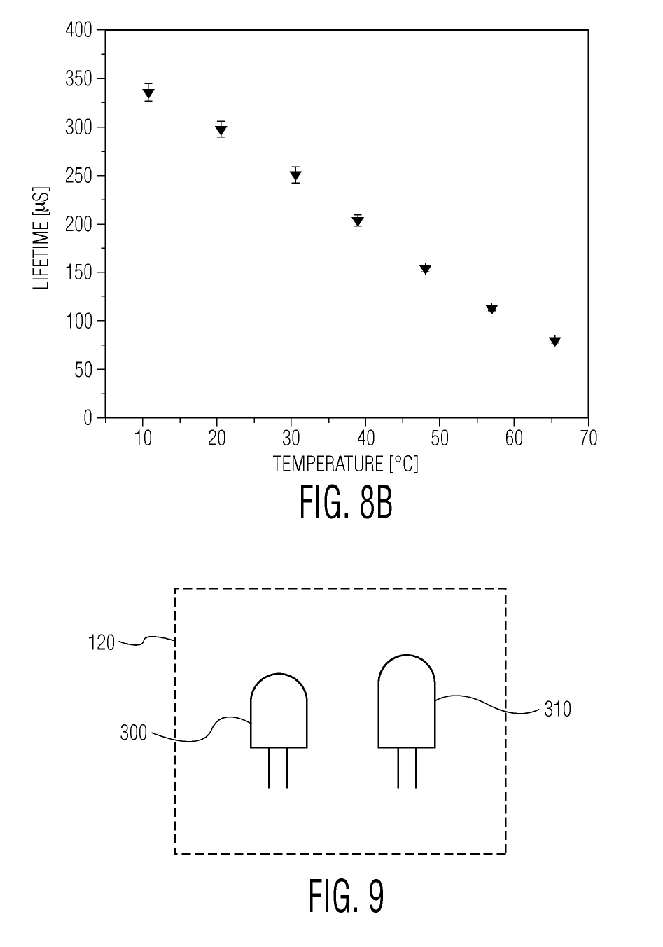

Unlike most organic dyes and the ruthenium complexes, the decay time of eutdap is unaffected by solvent or the presence of oxygen. The excitation band of eutdap is broad and can be found in the near UV and blue region, as shown in FIG. 7. However, the emission spectrum shows one dominant sharp peak at 613 nm and two minor bands at 580 and 590 nm, as shown in FIG. 8.

The luminescence of eutdap is strongly sensitive to temperature. FIGS. 7 and 8A show how the emission declines gradually as the temperature is increased from 10 to 70° C. The emission intensity is reduced by 75 percent within this temperature range. Similarly, the decay time of eutdap drops from 330 μs to 80 μs, as shown in FIG. 8B. The decrease in decay time can be fitted to a quadratic equation. The decay curve itself is double exponential term suggesting that the microenvironments of the dye molecules are not homogeneous.

All luminophores mentioned above are commercially available. Therefore their synthesis is not necessary. However, if higher purity is required the dyes can be purified by recrystallization. To entrap the dyes in PAN we there are two preferred approaches.

The first approach is to incorporate the luminophores in nanospheres, preferably oxygen impermeable polymer nanospheres. The preparation of polyacrylonitrile nanospheres in aqueous solution is based on the method described by Koese et al. (19). Specifically, 120 mg of PAN is dissolved in 25 mL of N,N-dimethylformamide (DMF). The luminophores, such as rubpy, are added to the PAN/DMF solution with vigorous stirring. The optimal concentration of rubpy for maximum luminescence is determined by adding different concentrations to the PAN.

In a separate beaker, 60 mg of SDS is dissolved in 125 mL of water, and transferred to a buret. Then, the SDS/water solution is added dropwise to the stirred DMF/PAN/rubpy solution. After the addition of approximately 7 mL of the aqueous SDS solution, the mixture should become opalescent because of the formation of nanoparticles. When the addition is complete, the solution is centrifuged and the residue washed sequentially with 100 mL of water and 50 mL of acetone. The nanoparticles are dried for at least 10 hours under vacuum at room temperature.

The second approach makes use of silica gel particles as adsorbent. The dyes are adsorbed on silica gel beads, which are then covered with a thin layer of PAN. In principle, this approach has several advantages over the first approach, although it requires more preparation steps.

The silica particles are a strong adsorbent. As such, they serve to immobilize the dye molecules on the surface, thereby reducing potential leaching. Further, due to the reflective surface of silica, the particles can function as a mirror to reflect the luminescence light rather than the luminescent light being absorbed by the skin.

These doped silica beads are preferably fabricated as follows. The luminophores are dissolved in an appropriate solvent, such as ethanol or acetone. The commercially available silica beads are tempered at 120° C. for at least 8 hours in order to activate their surface. The dye solution is then added to the silica, and the suspension stirred at room temperature overnight.

Afterwards, the suspension is centrifuged and the supernatant decanted. The silica with the adsorbed dyes is dried under vacuum at 50° C. for at least 12 hours. Meanwhile, the PAN/DMF solution (100 mg PAN, 50 mL DMF) is prepared. 0.5 g of the dried silica is added to this solution and the suspension is stirred for 10 minutes. The suspension is centrifuged and the supernatant removed.

Under vigorous stirring, 50 mL acetone is added to the silica gel and stirred for 30 minutes. Afterwards, the silica beads (now covered with a thin layer of PAN) are removed from the acetone by centrifugation. The silica beads are repeatedly washed with acetone to remove excess free dyes. The thickness of the PAN layer can be controlled by variation of the concentration of the PAN solution. Finally, the beads are dried under vacuum at room temperature for 12 hours. The immobilized dyes are now ready to be incorporated into the hydrogel.

### Hydrogel Matrix

As discussed above, there is a need for remote and non-adhesive temperature sensing for infants. To this end, the gel sensors 110 preferably utilize a skin-friendly vehicle or matrix for the luminophore dyes. The matrix in which the sensing luminophores are incorporated is a replacement for the harmful adhesive currently used to keep thermistors in place.

The matrix provides several important functions. It is the affixing component that enables contact of the luminophore dyes with the skin 210. It encapsulates the dyes within a semi-solid structure preventing the dyes from being blown off (if powder) or wiped off (if liquid) from the skin 210. The matrix also provides good heat transfer from the skin/body to the temperature sensing luminophore dyes, allowing for precise body temperature measurements to be carried out.

In order to be used as a temperature sensor on a human subject, the matrix should be biocompatible, should not harbor harmful microorganisms and should be non-irritating to the skin 210. Also, the matrix should not excessively absorb heat from the radiant warmer in the incubator or other sources, as this can cause a temperature reading higher than the skin temperature. Further, while the matrix should adhere well to the skin 210, it should be easily removable without irritating or harming the skin 210.

During the last two decades, significant advances have been made in the development of biocompatible and biodegradable materials for biomedical applications. In the biomedical field, the goal is to develop and characterize artificial materials for use in the human body to measure, restore, and improve physiologic function, and enhance survival and quality of life.

Typically, inorganic (metals, ceramics, and glasses) and polymeric (synthetic and natural) materials have been used for such items as artificial heart-valves, (polymeric or carbon-based) and synthetic blood-vessels. However, the preferred materials for system 200 are hydrogels.

Hydrogels are a network of polymer chains that are water-insoluble and are highly absorbent. They can contain over 99% water, which makes them highly flexible like natural tissue. Applications for hydrogels cover a wide range of fields. For example, polylactic acids are used as scaffolds in tissue engineering, 2-hydroxypropyl-methacrylate polymers (HPMA) has been widely employed in drug delivery systems, and polyacrylic acid is used as the super-absorbent in disposable diapers. In addition, contact lenses are made from silicone or polyacrylamide (15).

Of particular interest are hydrogels used for wound dressings, as they have the desired properties suitable for the sensitive skin of neonates. As such, they create or maintain a moist environment so that the skin 210 cannot dry out. They are well permeable to oxygen, so that the skin 210 underneath can breathe. In addition, these hydrogels protect wounds from the entry of microbes. Moreover, they adhere firmly even on the wounds, and can be gently removed without irritation.

Glyceryl polyacrylate (GPA) and chitosan are the subjects of active research as antibacterial wound dressing gels. Chitosan is a linear polysaccharide composed of randomly distributed β-(1-4)-linked D-glucosamine and N-acetyl-D-glucosamine produced commercially by de-acetylation of chitin, the structural element in the exoskeleton of crustaceans (crabs, shrimp, etc.) and cell walls of fungi. The amino group in chitosan has a pKa value of ˜6.5, thus, chitosan is positively charged and soluble in acidic to neutral solution with a charge density dependent on pH and the deacetylation value. This makes chitosan and its derivatives a bio-adhesive which readily binds to negatively charged surfaces, such as mucosal membranes.

Chitosan and its derivatives are approved to be hypoallergenic and antibacterial. Its high tensile and bioadhesive strength are advantageous for forming a tacky sensing layer. Further, it can be gently dissolved by a slightly acidic solution at pH 6.0.

Glyceryl polyacrylate (GP) is a clathrate gel known for its high water rentention ability. It does not dry even when exposed to ambient air or subjected to vacuum for 48 hours. This property is particularly useful for inactivating microbes by depriving them of water through osmotic effect. Studies show that by adding a certain amount of glycerol, the viscosity of the gel can be controlled. This results in a product that can adhere well on the skin 210 but that can also be easily peeled off (16).

The chitosan gel is preferably prepared as follows. 25 mL deionized water and 75 mL glycerol are mixed together, and the pH of the solution adjusted to 4. 1 g chitosan is added to the solution and stirred at room temperature for 2 hours. By then, the chitosan should be completely dissolved, resulting in a clear pale yellow solution. The dyed PAN beads are added to the solution and stirred for 10 minutes. Under continuous stirring, the solution is neutralized by the addition of monosodium phosphate. During this process, the solution gels and becomes clear. The gel is then ready to use.

The GP gel is preferably prepared as follows. 35 g glycerol, 10 mL deionized water and a measured amount of the sensing microbeads are mixed together. This mixture is added to 55 g glyceryl polyacrylate and stirred for three hours so that a homogeneous gel is formed.

The hydrogel matrix preferably includes oxygen radical scavengers in order to mitigate oxygen interference. Preferable oxygen radical scavengers include, but are not limited to, ascorbate (vitamin C) and tocopherol (vitamin E).

### Excitation, Detection and Analysis

Luminescence detection is an established technique used in various fields of science. Hence, a variety of detection systems have been developed. Bulky, highly sophisticated systems consisting of lasers as the excitation source and a fluorimeter as the detector are set up in laboratories for basic research purposes.

In the last two decades, low cost luminescence based oxygen, pH and CO₂ sensors have been successfully commercialized. Though low cost, these portable sensors are able to measure parameters accurately. They utilize inexpensive LEDs as the excitation light source and photodiodes as the photodetector, whereas table-top fluorimeters generally employ the more sensitive photomultiplier tubes.

Both kinds of photodetectors are meant for measurement of a focused, immobile luminescence source. Thus, they are not designed to locate the luminescence source emanating from a point on a larger non-luminescent object. In system 200, sensing a small spot on a baby's skin is a preferred capability.

Localization and measurement has been achieved previously for similar applications with the advancement of digital imaging systems. CCD and CMOS based cameras have become more sensitive and fast, so that they have been able to not only make luminescence images from samples with quantitative intensity, but also detect the decay time of the luminescent samples. These cameras have become an integral part of fluorescence microscopy, making it possible to localize bio-compounds in cells and improve the understanding of intramolecular processes.

Luminescence imaging is mostly used in combination with microscopy. However, there are a few research groups employing this technique for oxygen and temperature sensing. Hradil et al. developed a system comprising of a LED bank as the excitation source and an image-intensified gated CCD camera with cooling system and a matrix consisting of an oxygen sensitive ruthenium complex and a temperature sensitive magnesium fluorogermanate phosphor (17). With this system, they are able to measure oxygen and temperature simultaneously by determining the change in luminescence decay time with the concentration of oxygen and the temperature (17).

Baleiza et al. used a similar setup to determine oxygen and temperature (18). However, ruphen with a decay time of less than 4 μs (3 ms is the decay time of the fluorogermanate phosphor) is employed as the temperature probe (18). Köse et al. used a luminescence imaging system to measure oxygen and temperature by quantifying the luminescence intensity of ruphen (19). The luminescence intensity is then mathematically processed using the principle component analysis technique. This mathematical procedure is reported to improve the accuracy of the measurement significantly (19).

In the above applications, the sample to be imaged is static and shielded from ambient light, which makes the measurement relatively easy. However, measuring the temperature with an accuracy of better than 0.1° C. remotely is a more challenging task. Not only must the sensing chemistry be highly temperature sensitive, but the luminescence detection should be adapted to the conditions in the neoneate incubator.

Because of these challenges, pure intensity measurements are preferably not used in system 200. Instead, ratiometric measurements or decay time measurements are preferably used. The ratiometric method has the advantage that the imaging system, and the data acquisition and processing system need not be very sophisticated and fast. However, two luminescence signatures need to be measured to arrive at the ratio.

The conventional method requires two different optical band pass filters in order to isolate the two emission signatures. With a single camera, the filter placed in front of the lens has to be repeatedly changed, which is potentially a cumbersome process. However, automation is possible with a filter wheel attached to a controllable motor.

A preferred approach to discriminate between two signals, even when both excitation and emission of the two signals are identical, is a technique based on the large decay time difference between two luminophores. When a luminophore is exposed to intensity modulated excitation light, the observed luminescence intensity is dependent on the modulation frequency ω and the luminophore decay time τ. This dependency is described by the following equation: ƒ=s/(1+ω²*τ²)^{-0.5}, where s is the steady state luminescence intensity of the fluorophore, w is the angular modulation frequency and τ is the decay time.

According to this equation, the modulated luminescence decreases with increasing modulation frequencies, eventually reaching a certain frequency where the luminescence is completely diminished (i.e., demodulated) (20). The longer the decay time, the lower this boundary frequency is. Suppose there are two luminophores in a system with a decay time difference of three orders of magnitude. When the system is excited with modulated excitation light at a frequency where the luminescence of the dye with longer decay time is demodulated, only the luminescence of the dye with shorter decay time will be observed.

At a lower frequency, emission from both the dyes will be observed. Thus, one can calculate the ratio of the emission at the higher and lower excitation frequencies as an alternative to intensity ratios at two wavelengths. This technique has been successfully exploited for the determination of glucose and glutamine using fluorescently labeled periplasmic binding protein sensors (21).

Decay time measurements usually require sophisticated and fast performing imaging systems. Conventional methods use multi-channel plate (MCP) based image intensifiers. Unfortunately, MCPs are comparatively expensive, prone to photo-damage due to overexposure and require elaborate electronics. Moreover, MCP's spatial resolution is relatively low. Furthermore, MCPs can inject a comparatively high noise level in the measurement.

The controller 140 of system 200 preferably utilizes lock-in imaging for decay time measurements. This enables decay time measurements for a fraction of the cost and space of an MCP. As demonstrated by Wouters et al., the decay time can be determined by the phase modulation technique (22). In this approach, the luminescence modulated with a frequency ω is phase shifted in comparison to the excitation light. The phase shift φ is dependent on the decay time τ by the relation τ=tan(φ)/ω.

The phase shift can be determined with camera images as follows: Images are taken with phase delays (relative to the modulated excitation light) of 0, 0.5π, π and 1.5π. The phase shift is calculated as φ=arctan [S_{1.5π}−S_{0.5π})/(S0−S_{π})]−φ′, where φ′ is the instrumental phase delay which can estimated by measuring the reflected excitation light. This technique is immune to interference from ambient light, which ensures precise temperature measurements.

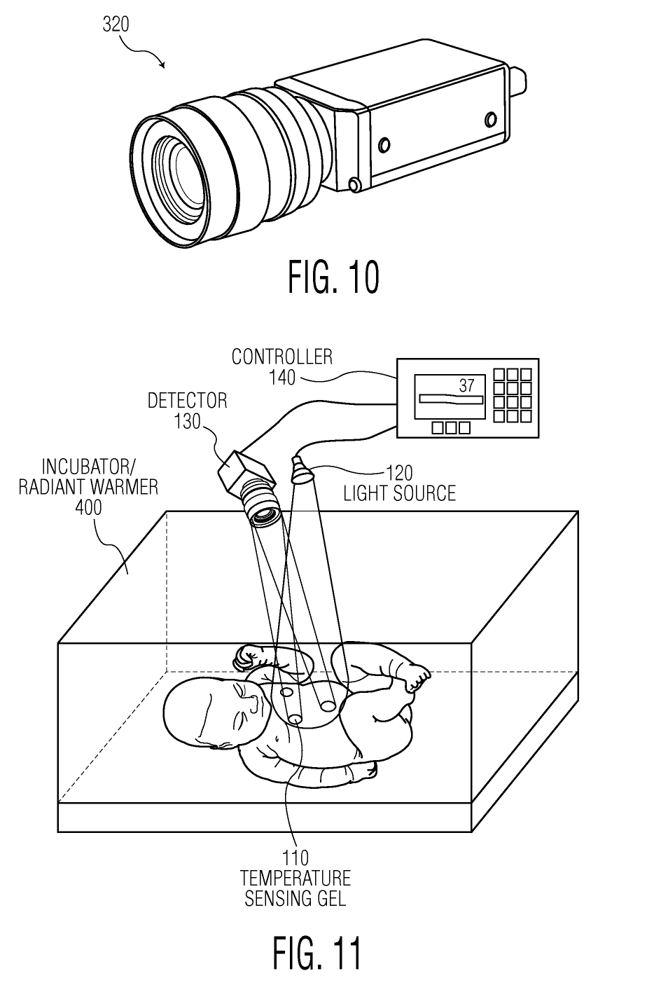

In system 200, the excitation light source 120 is preferably a high power blue LED 300 and a red LED 310, as shown in FIG. 9. However, other light sources can be used including, but not limited to, laser diodes. The detector 130 is preferably a CCD camera 320, such as the one shown in FIG. 10. However, other types of detectors can be used including, but not limited to, a photoresistor, a photodiode, an avalanche photodiode and a photomultiplier tube.

If a CCD camera is used, it preferably has an imaging speed of 90 fps at 640×480, and is externally triggerable. Trigger delay control preferably ranges from 0 to 60 s with 1 us increments. This will allow for images to be taken with satisfactory frequency and at the desired phase. The CCD camera and the LEDs are preferably equipped with appropriate band pass filters, and they are connected to the controller 140, which is programmed with controlling and image processing software.

The controller 140 modulates the LEDs 300 and 310 to a certain frequency that is dependent on the dye in use. In order to calculate the decay time of the luminophore correctly, the instrumental phase delay is determined. For that purpose, the red LED 310 is modulated. The modulation signal is created by the controller 140.

The image acquisition process is in sync with this modulation frequency. Parts of the modulated LED light 170 reflect from the skin 210 to the camera 320, which then takes images at different phases (22). The phase shift caused by the camera 320 and controller 140 is then calculated using the techniques described above.

As the instrumental phase shift does not change significantly for a long period of time, this measurement need not to be done for every temperature measurement cycle. Once the instrumental phase delay is known, the decay time measurement can be carried out. The image acquisition process is the same as for the instrumental phase delay measurement. However, instead of the red LED 310, the blue LED 300 is used for the excitation of the gel sensor 110.

The controller processes the acquired images. It identifies the luminescent spot and isolates the responsible pixels, while abandoning the rest of the image. The isolated images of the spot are then used for the calculation of the decay time. Decay time measurements are carried out repeatedly for better accuracy. Based on the decay time, the temperature can be calculated.

For decay time assisted ratiometric measurements, the procedure is simpler. First, a gel sensor 110 containing two dyes with different decay times is illuminated with modulated light at a certain frequency where the luminescence of the dye with longer decay time will be completely demodulated. Images are taken at a phase equal to zero. The luminescence is read from the image.

Next, the modulation frequency of the excitation light source 120 is decreased to a level at which both dyes are fully modulated. The images taken from the sensing spot contains the luminescence of both dyes. The luminescence ratio between the two measurements is used to calculate the temperature.

### Incorporation into Incubators and Radiant Warmers

As discussed above, the present invention is particularly suitable as a remote body temperature sensing system in incubators and radiant warmers 400 for infant and neonatal care, as shown in FIG. 11. Temperature measurements can be taken remotely without the need for the standard adhesive thermistors that can irritate or damage the baby's skin. The gel sensors 110 are easy to apply and remove without the risk of damaging the baby's skin. Further, the gel sensors 110 do not require a wired connection, thereby eliminating the risk of the baby getting tangled in wires. In addition, because the remote body temperature system can operate in the visible spectrum, there is no detector interference from the radiant warmers used in incubators and radiant warmers.

The foregoing embodiments and advantages are merely exemplary, and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art. Various changes may be made without departing from the spirit and scope of the invention, as defined in the following claims (after the Appendix below). For example, although the present sensor system has been described in connection with a remote body temperature sensing system, it can be adapted for the remote sensing of other parameters by choosing the appropriate light source and gel sensors.

### APPENDIX

1. Heim T. Energy requirements of thermoregulatory heat production in the newly born. In: Monset-Couchard M, Minkowski A, eds. Physiological and Chemical Basis for Perinatal Medicine. 1988:158-174 Karger Basel, Switzerland.
2. Dollberg S, Xi Y, Donnelly M. A noninvasive transcutaneous alternative to rectal thermometry for continuous measurement of core temperature in the piglet. Pediatr Res. 1993; 34:512-517
3. Dollberg S, Hoath S. Infant warming and temperature monitoring. Neonatal Intensive Care. 1995 Seattle, Wash.: Space Labs Medical, Inc.
4. Sauer P, Dane H, Visser H. New standards for neutral thermal environment of healthy very low birth weight infants. Arch Dis Child. 1984; 59:18-22.
5. Chandon S, Baumgart S. Temperature regulation of the premature infant. In Taeusch H W, Ballard R A, Gleason C, Avery M E, ed. Schaffer and Avery's diseases of the Newborn. 6^{th} ed. Philadelphia: Elsevier Health Sciences. 2004:364-371.
6. Baumgart S. Current concepts and clinical strategies for managing low-birth-weight infants under radiant warmers. Medical Instrumentation. 1967: 21(1): 23-26.
7. Dollberg S, Hoath S. Temperature regulation in preterm infants: Role of the skin-environment interface. NeoReviews. 2001: 2(12): e282-e290.
8. Okken A, Koch I, eds. Thermoregulation of the Sick and Low Birth Weight Neonate. 1995 Springer Berlin, Germany.
9. Khalil G., Lau K., Phelan G. D., Carlson B., Gouterman L., Callis J., and Dalton L., Europium beta-diketonate temperature sensors: Effects of ligands, matrix, and concentration, Review of Scientific Instruments, 2004, 75, 1
10. Zhang Z. Y., Zhang Z. Y., Grattan L. S., Fiber Optic Fluorescence Thermometry, Springer-Verlag New York, 1994, ISBN: 0412624702
11. Dobrucki J W, Interaction of oxygen-sensitive luminescent probes [Ru(phen)₃]²⁺ and [Ru(bipy)₃]²⁺ with animal and plant cells in vitro. Mechanism of phototoxicity and conditions for non-invasive measurements, Journal of photochemistry and photobiology. B, Vol. 65, 2-3, 136-44
12. Swarts J. W., Janssen A. E. M. and Boom R. M., (2008) Temperature effects during practical operation of microfluidic chips, Chemical Engineering Science, Volume 63, Issue 21, 5252-5257
13. Hartmann P., Leiner M., and Kohlbacher P., (1998) Photobleaching of a ruthenium complex in polymers used for oxygen optodes and its inhibition by singlet oxygen quenchers, Sensors and Actuators B: Chemical Volume 51, Issues 1-3, 31, 196-202
14. Andrew Mills A., Tommons C., Bailey R., Tedford M. and Crillyb P., (2006), Luminescence temperature sensing using poly(vinyl alcohol)-encapsulated [Ru(bpy)₃]²⁺ films, Analyst, 131, 495-500
15. Chiellini E., Sunamoto J., Migliaresi G., Ottenbrite R. and Cohn D., (2001) Biomedical Polymers and Polymer Therapeutics, Springer; 1 edition, ISBN-10: 0306464721
16. Woller W., Jones D., Hydrogel PolymerWound Dressing, May 11 1999, U.S. Pat. No. 5,902,600
17. Hradil J., Davis C., Mongey K., McDonagh C. and MacCraith B., (2002) Temperature-corrected pressure-sensitive paint measurements using a single camera and a dual-lifetime approach, Meas. Sci. Technol. 13 1552-1557
18. Baleiza C., Nagl S., Schaeferling M., Berberan-Santos M., Wolfbeis O., (2008) Dual Fluorescence Sensor for Trace Oxygen and Temperature with Unmatched Range and Sensitivity, Anal. Chem., 80, 6449-6457
19. Kose M., Carroll B., and Schanze K., (2005) Preparation and Spectroscopic Properties of Multi-Luminophore Luminescent Oxygen and Temperature Sensor Films, Langmuir, 21 (20), 9121-9129
20. Lacowicz J., (2006) Principle of Fluorescence Spectroscopy, Springer; 3rd edition, ISBN-10: 0387312781
21. Lam H., Kostov Y., Rao G., Tolosa L., (2008) Low-cost optical lifetime assisted ratiometric glutamine sensor based on glutamine binding protein, Analytical Biochemistry 383, 61-67
22. Esposito A., Oggier T., Gerritsen H. C., Lustenberger F., Wouters F. S., All-solid-state lock-in imaging for wide-field fluorescence lifetime sensing, OPTICS EXPRESS, 13, 24
23. Stücker M., Struk A., Altmeyer P., Herde M., Baumgärtl H. and Lübbers D., (2002) The cutaneous uptake of atmospheric oxygen contributes significantly to the oxygen supply of human dermis and epidermis, Journal of Physiology, 538, 985-994.
24. Cilurzo F., Minghetti P., and Sinico Ch., (2007) Newborn Pig Skin as Model Membrane in In Vitro Drug Permeation Studies: A Technical Note, AAPS PharmSciTech, 8 (4) Article 94
25. Kendra D F, Hadwiger L A. (1984) Characterisation of the smallest chitosan oligomer that is maximally antifungal to Fusarium solani and elicits pisatin formation in Pisium atvium. Experimental Mycology 8:276-81
26. Sekiguchi S., Molecular weight dependency of antimicrobial activity by chitosan oligomers. Food hydrocolloids: structures, properties, and functions. New York, USA: Plenum Press; 1994.71-6.
27. Greff D., Cosmetic, Dermopharmaceutical or Veterinary Composition For Desinfecting Human or Animal Skin, U.S. Pat. No. 6,123,953, Sep. 26, 2000
28. Wong C F, Yuen K H, Peh K K. (1999) Formulation and evaluation of controlled release Eudragit buccal patches. Int J. Pharm., 178:11 Y22.
29. Shabbir Bambot, Raja Holavanahali, Joseph Lakowicz, Gary Carter and Govind Rao. Phase Fluorometric Sterilizable Optical Oxygen Sensor. Biotechnol. Bioeng. 1994. 43:1139-1145.
30. Shabbir Bambot, Jeffrey Sipior, Joseph Lakowicz and Govind Rao. Lifetime-Based Optical Sensing of pH Using Resonance Energy Transfer in Sol-Gel Sensors. Sensors and Actuators B (Chemical). 1995. 22:181-188.
31. Jeffrey Sipior, Shabbir Bambot, Romauld M., Gary Carter, Joseph Lakowicz and Govind Rao. A Lifetime-Based Optical CO2 Gas Sensor with Blue or Red Excitation and Stokes or Anti-Stokes Detection. Anal. Biochem. 1995. 227:309-318.
32. Raja Holavanahali, Romauld M., Gary M. Carter, Govind Rao, Jeffrey Sipior, Joseph R. Lakowicz and John Bierlein. A Directly-Modulated Diode Laser Frequency-Doubled in a KTP Waveguide as an Excitation Source for CO2 and O2 Phase Fluorometric Sensors. J. Biomed. Optics. 1996. 1:124-130.
33. Jeffery Sipior, Lisa Randers-Eichhorn, Joseph Lakowicz, Gary Carter and Govind Rao. A Phase Fluorometric Optical CO2 Gas Sensor for Fermentation Off-Gas Monitoring. Biotech. Prog. 1996. 12:266-271.
34. Lisa Randers-Eichhorn, Roscoe Bartlett, Douglas Frey and Govind Rao. Non-Invasive Oxygen Measurements and Mass Transfer Limitations in Tissue Culture Flasks. Biotechnol. Bioeng. 1996. 51:466-478.
35. Zakir Murtaza, Qing Chang, Govind Rao and Joseph R. Lakowicz. Long Lifetime Metal-Ligand pH Probes. 1997. Analytical Biochemistry. 247:216-222.
36. Chang, Q., Randers-Eichhorn, L., Lakowicz, J. R., and Rao, G. A steam sterilizable fluorescence lifetime-based sensor for dissolved carbon dioxide. 1998. Biotechnology Progress. 14: 326-331
37. Ignacy Gryczynski, Zygmunt Gryczynski, Govind Rao and Joseph R. Lakowicz. Polarization-Based Oxygen Sensor. 1999. Analyst. 124:1041-1044.
38. Yordan Kostov, Peter Harms, Robert S. Pilato and Govind Rao. Ratiometric oxygen sensing: detection of dual-emission ratio through a single emission filter. 2000. Analyst. 125:1175-1178.
39. Kostov Y., Van Houten, K. A., Harms, P., Pilato, R. S., Rao., G. ((2000) A Unique Oxygen Analyzer Combining a Dual Emission Probe and a Low-Cost Solid-State Ratiometric Fluorometer. Appl. Spectroscopy. 54:864-868.
40. Kostov Y., Harms P., Randers-Eichhorn L., Rao G. Low-cost microbioreactor for high throughput bioprocessing. Biotechnol. Bioeng. 2001. 72, 346-352. *Won the Gaden Award for the Most Influential Paper of 2001 Published in Biotechnology & Bioengineering.
41. Kostov Y., Harms P. and Rao G. Ratiometric Sensing Using Dual Frequency Lifetime Discrimination. 2001. Anal. Biochem. 297:105-108
42. Kostov, Y., Gryczynski, I. and Rao, G. Polarization Oxygen Sensor: A Template for a New Class of Fluorescence-Based Sensors. 2002. Anal. Chem. 74, 2167-2171.
43. Haley Kermis, Yordan Kostov, Peter Harms and Govind Rao. Dual Excitation Ratiometric Fluorescent pH Sensor for Non-invasive Bioprocessing: Development and Application. 2002. 18:1047-1053. Biotech. Prog.
44. Yordan Kostov and Govind Rao. Ratio measurements in oxygen determinations: wave length ratiometry, lifetime discrimination and polarization detection. 2003. Sensors & Actuators B. 90:139-142
45. Ge, X., Yordan Kostov and Govind Rao. High-Stability Non-Invasive Autoclavable Naked Optical CO2 Sensor. 2003. Biosensors and Bioelectronics. 18:857-865.

What is claimed is:

1. A remote sensor for measuring a parameter of a system, comprising:
a light source for generating excitation light;
a gel sensor in physical communication with the system and positioned to receive the excitation light, wherein the gel sensor emits emission light in response to the excitation light and wherein a chemical property of the gel sensor is such that at least one characteristic of the emission light varies as a function of variations in the parameter being measured;
a detector for detecting the emission light from the gel sensor and for outputting a detector signal based on the detected emission light; and
a controller for receiving and analyzing the detector signal and for deriving a parameter value based on the detector signal.

2. The remote sensor of claim 1, wherein the gel sensor comprises a hydrogel matrix.

3. The remote sensor of claim 1, wherein the parameter comprises temperature.

4. The remote sensor of claim 3, wherein the gel sensor comprises temperature sensitive luminophores in a hydrogel matrix.

5. The remote sensor of claim 4, wherein the temperature sensitive luminophores comprise ruthenium(II) tris(1,10-phenanthroline) (ruphen), ruthenium(II) tris(bipyridine) (“rubpy”) or Tris-(dibenzoylmethane)mono(5-amino-1,10-phenanthroline)-europium(III).

6. The remote sensor of claim 4, wherein the temperature sensitive luminophores are encapsulated in an oxygen impermeable polymer.

7. The remote sensor of claim 6, wherein the oxygen impermeable polymer comprises polyacrylonitrile or silicone.

8. The remote sensor of claim 4, wherein the hydrogel matrix comprises glyceryl polyacrylate or chitosan.

9. The remote sensor of claim 1, wherein the light source emits excitation light within the visible spectrum.

10. The remote sensor of claim 1, wherein the light source comprises at least one light emitting diode.

11. The remote sensor of claim 1, wherein the detector comprises a CCD camera.

12. The remote sensor of claim 8, wherein the hydrogel matrix further comprises oxygen radical scavengers.

13. The remote sensor of claim 12, wherein the oxygen radical scavengers comprise ascorbate.

14. The remote sensor of claim 12, wherein the oxygen radical scavengers comprise tocopherol.

15. A system for remotely monitoring body temperature, comprising:
a light source for generating excitation light;
at least one gel sensor in physical contact with the body and positioned to receive the excitation light, wherein the at least one gel sensor emits emission light in response to the excitation light and wherein a chemical property of the at least one gel sensor is such that at least one characteristic of the emission light varies as a function of temperature;
a detector for detecting the emission light from the at least one gel sensor and for outputting a detector signal based on the detected emission light; and
a controller for receiving and analyzing the detector signal and for deriving a body temperature based on the analysis.

16. The system of claim 15, wherein the at least one gel sensor is in physical contact with skin.

17. The system of claim 15, wherein the at least one gel sensor comprises a hydrogel matrix.

18. The system of claim 15, wherein the at least one gel sensor comprises temperature sensitive luminophores in a hydrogel matrix.

19. The system of claim 18, wherein the temperature sensitive luminophores comprise ruthenium(II) tris(1,10-phenanthroline) (ruphen), ruthenium(II) tris(bipyridine) (“rubpy”) or Tris-(dibenzoylmethane)mono(5-amino-1,10-phenanthroline)-europium (III).

20. The system of claim 18, wherein the temperature sensitive luminophores are encapsulated in an oxygen impermeable polymer.

21. The system of claim 20, wherein the oxygen impermeable polymer comprises polyacrylonitrile or silicone.

22. The system of claim 18, wherein the hydrogel matrix comprises glyceryl polyacrylate or chitosan.

23. The system of claim 15, wherein the light source emits excitation light within the visible spectrum.

24. The system of claim 15, wherein the light source comprises at least one light emitting diode.

25. The system of claim 15, wherein the light source comprises at least one laser diode.

26. The system of claim 15, wherein the detector comprises a CCD camera.

27. The system of claim 26, wherein the controller adjusts a position of the CCD camera in response to movement of the at least one gel sensor.

28. The system of claim 15, wherein the at least one gel sensor generates emission light via luminescence.

29. The system of claim 28, wherein the controller derives the body temperature based on a steady state luminescence intensity of the emission light.

30. The system of claim 28, wherein the controller derives the body temperature based on ratiometric intensity measurements.

31. The system of claim 28, wherein the controller derives the body temperature based on a decay time of the luminescence.

32. The system of claim 15, wherein the at least one gel sensor is positioned inside an incubator, and the light source, detector and controller are positioned outside the incubator.

33. The system of claim 15, wherein the at least one gel sensor is positioned inside a radiant warmer, and the light source, detector and controller are positioned outside the radiant warmer.

34. The system of claim 15, wherein the detector comprises a photoresistor, a photodiode, an avalanche photodiode or a photomultiplier tube.

35. The system of claim 22, wherein the hydrogel matrix further comprises oxygen radical scavengers.

36. The remote sensor of claim 35, wherein the oxygen radical scavengers comprise ascorbate.

37. The remote sensor of claim 35, wherein the oxygen radical scavengers comprise tocopherol.
